(19) United States
(12) Patent Application Publication
(10) Pub. No.: US 2023/0380676 A1
(43) Pub. Date: Nov. 30, 2023

(54) MEDICAL DEVICE INCLUDING A TUBE

(71) Applicant: AMBU A/S, Ballerup (DK)

(72) Inventors: Jesper Domino RASK, Copenhagen S (DK); Martin Lund STØRUP, Brønshøj (DK)

(73) Assignee: AMBU A/S, Ballerup (DK)

(21) Appl. No.: 18/027,568

(22) PCT Filed: Aug. 23, 2021
(86) PCT No.: PCT/EP2021/073279

§ 371 Date: Mar. 21, 2023

(30) Foreign Application Priority Data
Sep. 22, 2020 (DK) PA 2020 70621

(51) Int. Cl. A61B 1/267; A61B 1/00; A61B 1/05; A61M 16/04; A61B 1/12

(52) U.S. Cl. CPC A61B 1/2676; A61B 1/00096; A61B 1/051; A61M 16/0459; A61M 16/0486; A61B 1/126; A61M 2207/00

(57) ABSTRACT

A main tube (2) has a first lumen (3) with a proximal end (5) and a distal end (6) to be placed inside a body cavity. An outer wall (7) encloses the first lumen and a dedicated camera lumen (4). A camera module is arranged at the distal end of the first lumen. An image transmission cable attached to the camera module extends through the camera lumen to connect to an image display device. A separately manufactured tip housing (20) is mounted on the main tube at the distal end of the first lumen. The tip housing forms a tip extension (23) of the first lumen and a tip extension (24) of the camera lumen. The tip extension of the first lumen has an open end (25), and the camera module is arranged at least partly in the tip extension of the camera lumen.

### CROSS-REFERENCE TO RELATED APPLICATIONS

This application is a National Phase entry under 35 U.S.C. § 371 of International Application No. PCT/EP2021/073279, filed Aug. 23, 2021, which claims the benefit of and priority from Denmark Patent Application No. PA 2020 70621, filed Sep. 22, 2020; said applications are incorporated by reference herein in their entirety.

### TECHNICAL FIELD

The present disclosure relates to a medical device in the form of an airway device including a main tube having at least a first lumen and a dedicated camera lumen, the first lumen having a proximal end and a distal end, the distal end being adapted to be placed inside a body cavity during use of the medical device, the tube having an outer wall enclosing at least the first lumen and the camera lumen, the camera lumen having a proximal end and a distal end, a camera module including at least an image sensor and a light source being arranged at the distal end of the first lumen, and an image transmission cable attached to the camera module extending through at least part of the camera lumen in order to connect to an image display device.

### BACKGROUND

EP 2 754 384 B1 (Ambu A/S) discloses an endobronchial tube which contains an integrated camera and light source and a cleaning nozzle arrangement disposed within a dedicated peripheral lumen within the tube's wall. The endobronchial tube may for instance be made of plastic, rubber, polymers or silicone. In the illustrated embodiment, the endobronchial tube is a double lumen tube including a tracheal lumen and a bronchial lumen. The camera is arranged in said peripheral lumen of the tube at the distal end of the tracheal lumen in order to visualise the tracheal carina and the distal end of the bronchial lumen during its insertion into the left or right bronchus. However, in known solutions, the distal end of the double lumen tube is manually cut, and the distal ends of the flushing lumens are manually sealed. In order to provide the cleaning nozzle arrangement, channels pointing towards the camera lens or towards a window arranged in front of the camera lens and connected to the flushing lumens are formed manually in the double lumen tube at the distal end thereof. Furthermore, the fitting of a camera module in said dedicated peripheral lumen may be cumbersome. In a manual procedure, firstly, said peripheral lumen may be dilated in order to fit the camera module, and, secondly, the camera module may be fixed in the lumen by means of glue. Following this procedure, correct positioning of the camera module in the peripheral lumen may be difficult.

### SUMMARY

The object of the present disclosure is to provide a medical device being easy to produce and assemble correctly.

In view of this object, a separately manufactured tip housing has a proximal end and a distal end, the proximal end of the tip housing is mounted on the main tube at the distal end of the first lumen, the tip housing forms a tip extension of the first lumen and a tip extension of the camera lumen, the tip extension of the first lumen has an open end, and the camera module is arranged at least partly in the tip extension of the camera lumen.

In this way, by arranging the camera module in a tip housing mounted as an extension on the main tube, the tip housing may be manufactured separately by other manufacturing techniques than that of the main tube, thereby forming the tip extension of the camera lumen precisely for fast, easy and correct positioning of the camera module, and thereby forming other necessary details, such as flushing channels, without manual adaptation during assembly.

A more precise camera mounting in terms of translational and rotational positioning may lead to less variance in camera angle between different devices, and thereby less variance in the view of the patient's important landmarks in the airway, as seen from an operator perspective. In general, improved quality and consistency of the final product may be achieved as a result of less operator dependency during production of the device.

The main tube is preferably manufactured by extrusion.

In an embodiment, the main tube has a flush lumen enclosed by the outer wall, the flush lumen has a proximal end and a distal end, the tip housing forms a tip extension of the flush lumen, and the tip extension of the flush lumen communicates with at least one flush outlet directed at a lens end of the camera module. Thereby, by forming the tip extension of the flush lumen in the tip housing, nozzles may be more precisely configured as compared to manually cut nozzles in existing solutions, thereby resulting in better performance when cleaning the camera front. For instance, if a patient has a lot of secretion in the airway, this can get in front of the camera. In this case, the flush outlet may be used to wash and/or blow away the secretion to regain a clear picture.

In an embodiment, the camera module is fixed in the tip extension of the camera lumen by means of a camera housing, and the at least one flush outlet forms part of the camera housing. Thereby, by arranging the flush outlet or outlets as an integrated part of the camera housing, an even more efficient cleaning of the camera front may be obtained as a result of close and precise positioning of the flush outlet in relation to a front lens of the camera or a transparent wall behind which a camera lens stack is arranged.

In an embodiment, the tip extension of the flush lumen is provided with a protruding pipe engaging the flush lumen of the main tube. Thereby, a continuous lumen without blockage in the junction may be ensured. The protruding pipe may be manufactured as an integrated part of the tip housing or may be a separate pipe component inserted into the tip housing.

In an embodiment, the main tube is provided with a first inflatable cuff, the main tube has a first cuff lumen enclosed by the outer wall and communicating with the first inflatable cuff, the first cuff lumen has a proximal end and a distal end, and the distal end of the first cuff lumen is closed by means of a sealing plug arranged on the tip housing. Thereby, a first inflatable cuff may be arranged on the main tube without having to manually close the distal end of the first cuff lumen during production. The sealing plug may be a separate part arranged on the tip housing or it may be an integral part of the tip housing.

In an embodiment, the main tube has an anesthetic lumen enclosed by the outer wall, the anesthetic lumen has a proximal end and a distal end, the tip housing forms a tip extension of the anesthetic lumen, and the tip extension of the anesthetic lumen communicates with at least one anesthetic outlet port. Thereby, the at least one anesthetic outlet port may be precisely formed during manufacturing of the tip housing without manual adaptation during production of the device.

In an embodiment, the tip extension of the anesthetic lumen is provided with a protruding pipe engaging the anesthetic lumen of the main tube. Thereby, a continuous lumen without blockage in the junction may be ensured. The protruding pipe may be manufactured as an integrated part of the tip housing or may be a separate pipe component inserted into the tip housing.

In an embodiment, the main tube has a second lumen having a proximal end and a distal end, the second lumen is enclosed by the outer wall, and the tip housing forms a tip extension of the second lumen.

In an embodiment, a distal tube is mounted on the distal end of the tip housing, thereby forming a further extension of the second lumen. Thereby, the distal tube may have different properties, such as hardness, stiffness, etc., than the main main tube and/or the tip housing whereby more design freedom may be achieved. The distal tube may preferably be an extruded tube. For instance, the distal tube may be more flexible than the main tube and may at least be more flexible than the tip housing. However, alternatively, the distal tube could be replaced by an integrated part of the tip housing. Such integrated part of the tip housing and the tip housing itself could be produced together from two different materials, respectively, by a co-injection moulding technique, whereby the materials have different properties, such as different flexibilities.

In an embodiment, the distal tube is provided with a second inflatable cuff, the main tube has a second cuff lumen enclosed by the outer wall, the tip housing forms a tip extension of the second cuff lumen, the distal tube forms a distal second cuff lumen enclosed by an outer wall of the distal tube, and the distal second cuff lumen of the distal tube connects the tip extension of the second cuff lumen and the second inflatable cuff.

In an embodiment, the tip extension of the second cuff lumen is provided with a first protruding pipe arranged at the proximal end of the tip housing and engaging the second cuff lumen of the main tube, and the tip extension of the second cuff lumen is provided with a second protruding pipe arranged at the distal end of the tip housing and engaging the distal second cuff lumen of the distal tube. Thereby, a continuous lumen without blockage in the junctions may be ensured. The protruding pipes may be manufactured as integrated parts of the tip housing or may be separate pipe components inserted into the tip housing.

In an embodiment, the camera module is fixed in the tip extension of the camera lumen by means of a camera housing extending in a longitudinal direction and having a proximal end and a distal end, the camera housing includes a tubular housing part at least partly surrounding the camera module and a distal end wall, the tubular housing part and the distal end wall are integrally moulded and form one single housing element, and the tubular housing part fits tightly into the tip extension of the camera lumen of the tube. Thereby, precise mounting of a camera module in the tip extension of the camera lumen may be facilitated in that a tight fit between the tubular housing part and the camera lumen may ensure precise positioning of the camera module in the lumen without any adjustments being necessary.

In an embodiment, the tip housing is made by moulding, such as injection moulding, or 3D printing. Thereby it may be ensured that the tip extension of the camera lumen may be formed precisely for fast, easy and correct positioning of the camera module, and thereby forming other necessary details, such as flushing channels, without manual adaptation during assembly.

In an embodiment, the proximal end of the tip housing has a proximal junction interface fitting an abutment surface of the main tube, said abutment surface includes at least an end face of the outer wall of the main tube, the proximal junction interface forms a groove with said abutment surface of the main tube, and said groove contains hardened glue. Thereby, a secure connection between the proximal end of the tip housing and the main tube may be ensured.

In an embodiment, the tip extension of the at least first lumen, at the proximal end of the tip housing, is provided with a protruding pipe engaging the at least first lumen of the main tube. Thereby, a continuous lumen without blockage in the junction may be ensured. The protruding pipe may be manufactured as an integrated part of the tip housing or may be a separate pipe component inserted into the tip housing.

In an embodiment, the distal end of the tip housing has a distal junction interface fitting an abutment surface of the distal tube, said abutment surface includes at least an end face of the outer wall of the distal tube, the distal junction interface forms a groove with said abutment surface of the distal tube, and said groove contains hardened glue. Thereby, a secure connection between the distal end of the tip housing and the distal tube may be ensured.

In an embodiment, the tip extension of the second lumen, at the distal end of the tip housing, is provided with a protruding pipe engaging the further extension of the second lumen formed in the distal tube. Thereby, a continuous lumen without blockage in the junction may be ensured. The protruding pipe may be manufactured as an integrated part of the tip housing or may be a separate pipe component inserted into the tip housing.

In an embodiment, the medical device is a catheter.

In an embodiment, the camera module is fixed in the tip extension of the camera lumen.

In an embodiment, the medical device is an endotracheal tube.

In an embodiment, the system includes a monitor or image display device connected to the image transmission cable.

### BRIEF DESCRIPTION OF THE DRAWINGS

The disclosure will now be explained in more detail below by means of examples of embodiments with reference to the very schematic drawing, in which

FIG. 1 is a perspective view of a prior art double lumen endotracheal tube;

FIG. 2 illustrates, on a larger scale, a detail of FIG. 1;

FIG. 3 is a perspective view of a prior art single lumen endotracheal tube;

FIG. 4 is a perspective view of a double lumen endotracheal tube according to the present disclosure;

FIG. 5 is a perspective view of a single lumen endotracheal tube according to the present disclosure;

FIG. 6 is a side view of the single lumen endotracheal tube of FIG. 5;

FIG. 7 is an exploded sectional view taken along the line VII-VII of FIG. 6;

FIG. 8 shows, on a larger scale, the detail VIII of FIG. 7;

FIG. 9 is a top view of the single lumen endotracheal tube of FIG. 5;

FIG. 10 is an exploded sectional view taken along the line X-X of FIG. 9;

FIG. 11 shows, on a larger scale, the detail XI of FIG. 10;

FIG. 12 is a perspective view of a tip housing of the single lumen endotracheal tube of FIG. 5;

FIG. 13 is an exploded side view of the double lumen endotracheal tube of FIG. 4;

FIG. 14 is an exploded sectional view taken along the line XIV-XIV of FIG. 13;

FIG. 15 shows, on a larger scale, the detail XV of FIG. 14;

FIG. 16 is another side view of the double lumen endotracheal tube of FIG. 4, indicating a cross-section taken along the line XVII-XVII;

FIG. 17 is an exploded sectional view taken along the line XVII-XVII of FIG. 16;

FIG. 18 is a side view corresponding to that of FIG. 16 of the double lumen endotracheal tube of FIG. 4, indicating a cross-section taken along the line XIX-XIX;

FIG. 19 is an exploded sectional view taken along the line XIX-XIX of FIG. 18;

FIG. 20 is a side view of a tip housing of the double lumen endotracheal tube of FIG. 4;

FIG. 21 is an end view of the tip housing of FIG. 20, seen from the right side of the figure;

FIG. 22 is an end view of the tip housing of FIG. 20, seen from the left side of the figure;

FIG. 23A to 23D are schematic illustrations, seen in axial cross-section, of possible connections between the tip housing and a main tube of the endotracheal tubes of FIGS. 4 and 5; and

FIG. 24 is a perspective view of an embodiment of an assembled camera housing for the endotracheal tubes of FIGS. 4 and 5.

In the following description, corresponding elements of illustrated prior art devices as well as of described embodiments of the present disclosure, are denoted by corresponding reference numerals.

### DETAILED DESCRIPTION

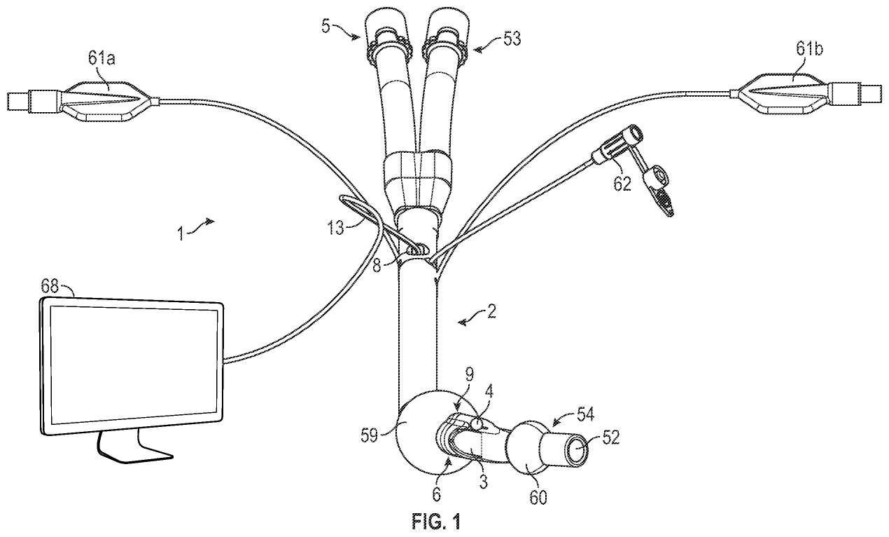

FIG. 1 illustrates a prior art medical device 1 in the form of a double lumen endotracheal tube 2 having a first lumen 3 in the form of a tracheal lumen and a second lumen 52 in the form of a bronchial lumen. A double lumen endotracheal tube as such is a well-known medical device and the use thereof is well-known to the skilled person. The first lumen 3 and the second lumen 52 extend together from the proximal end 5 of the first lumen 3 to the distal end 6 of the first lumen 3, and the second lumen 52 extends further from the distal end 6 of the first lumen 3 to a distal end 54 of the second lumen 52. The double lumen endotracheal tube has a first inflatable cuff 59 arranged proximally the open distal end 6 of the first lumen 3 and a second inflatable cuff 60 arranged proximally the open distal end 54 of the second lumen 52. Each of the first and second inflatable cuffs 59, 60 may be inflated in a well-known manner via a pilot balloon 61a, 61b provided with a one-way valve.

In use, the first inflatable cuff 59 is arranged in the trachea and the second inflatable cuff 60 is arranged in the left or right bronchus. Thereby, each bronchus may be ventilated separately in a well-known manner. The endotracheal tube may for instance be made of plastic, rubbers, e.g. butyl rubber, natural rubber or nitrile rubber, latex, neoprene, isoprene, polymers or silicones. The endotracheal tube may be made of Poly vinyl chloride (PVC) (with plasticizer), Thermoplastic elastomer (TPE), e.g. Styrene ethylene butylene styrene (SEBS), Styrene butylene styrene (-SBS), Thermoplastic Polyurethanes (TPU) or Thermoplastic vulcanizates (TPV). Most TPVs are binary blends of polyolefins and thermoplastic diene elastomers. Other elastomers sometimes used in TPVs include butyl rubber, natural rubber or nitrile rubber blended with iPP. Alternatively, blends of one or more of these materials may be used for producing the medical device, such as a catheter or endotracheal tube.

The endotracheal tube may be a single use product. The different lumens may be co-extruded in the tube 2.

The endotracheal tube 2 furthermore includes a camera module arranged and preferably fixed in a dedicated camera lumen 4 at a distal end 9 thereof. The camera module is arranged in said lumen of the tube 2 at the distal end of the tracheal lumen 3 in order to visualise the tracheal carina and the distal end of the bronchial lumen during its insertion into the left or right bronchus.

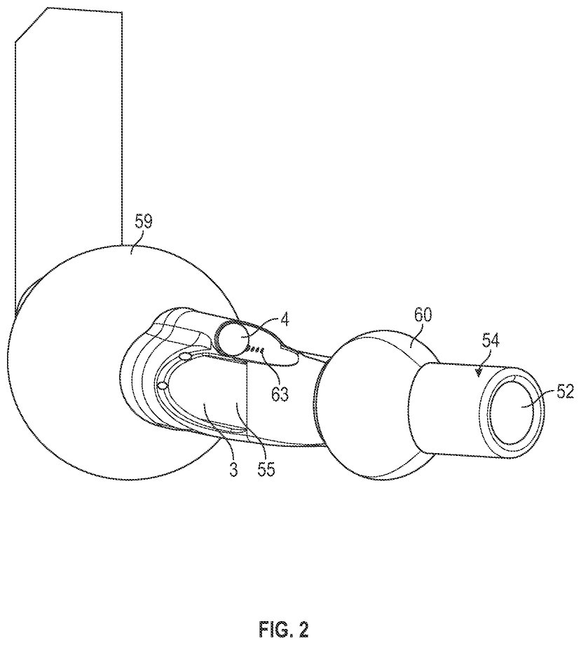

Furthermore, by means of a flush connection 62, a distal end wall of a camera housing including the camera module may be flushed via flush openings 63 formed in the tube 2, as best seen in FIG. 2. The flush connection 62 is connected to the flush openings 63 via a number of not illustrated flush channels co-extruded in an outer wall of tube 2.

In the prior art double lumen endotracheal tube 2 illustrated in FIGS. 1 and 2, the distal end of the double lumen tube has been manually cut, and the distal ends of the flushing lumens have been manually sealed. In order to provide the cleaning nozzle arrangement, the channels pointing towards the camera lens or a window in front of the camera lens and connected to the flushing openings 63 have been formed manually in the double lumen tube at the distal end thereof. The fitting of a camera module in the dedicated peripheral lumen 4 may be cumbersome. In a manual procedure, firstly, said peripheral lumen 4 may be dilated in order to fit the camera module, and, secondly, the camera module may be fixed in the lumen by means of glue. Following this procedure, correct positioning of the camera module in the peripheral lumen may be difficult.

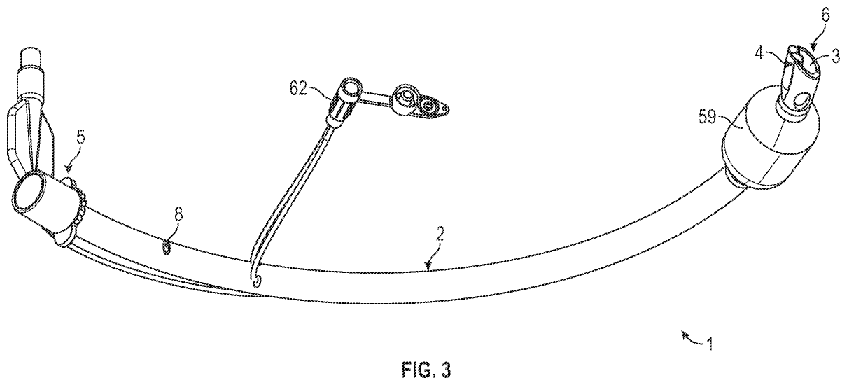

FIG. 3 illustrates another prior art medical device 1 in the form of a single lumen endotracheal tube 2.

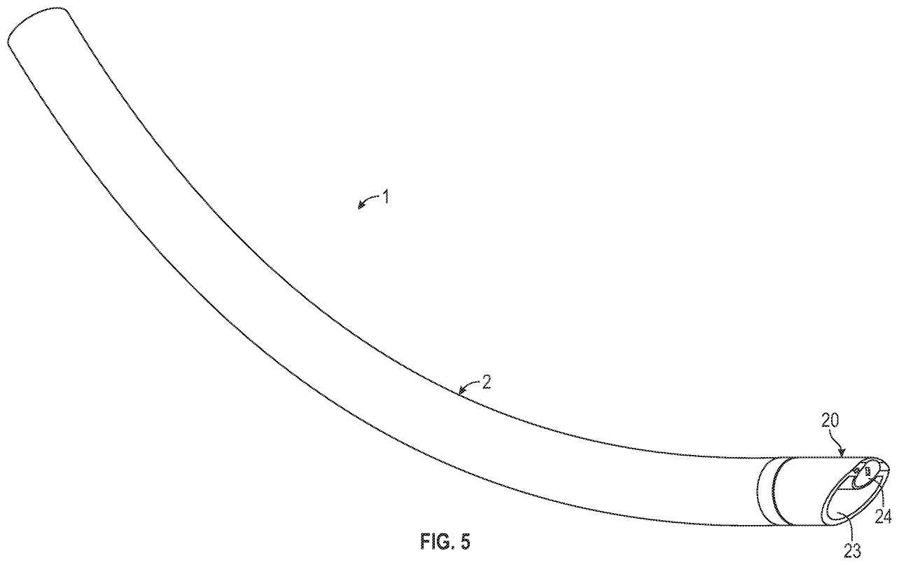

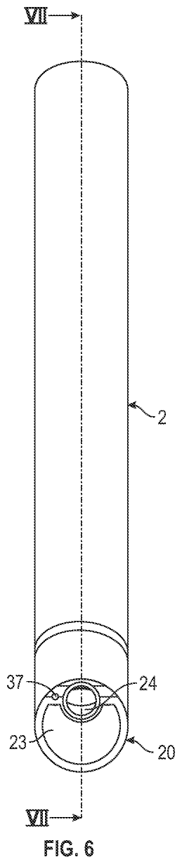

In an embodiment of the present disclosure, the tube 2 of the prior art medical device 1 illustrated in FIG. 3 has been replaced by a combination of a main tube 2 and a tip housing 20 as illustrated in FIGS. 5 to 12. The main tube 2 is preferably an extruded tube.

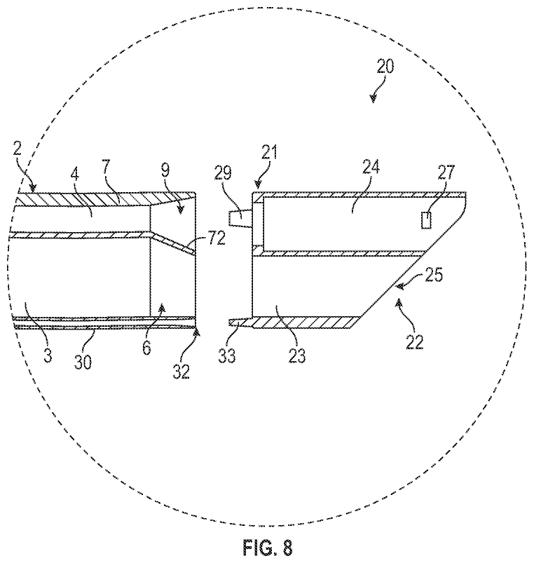

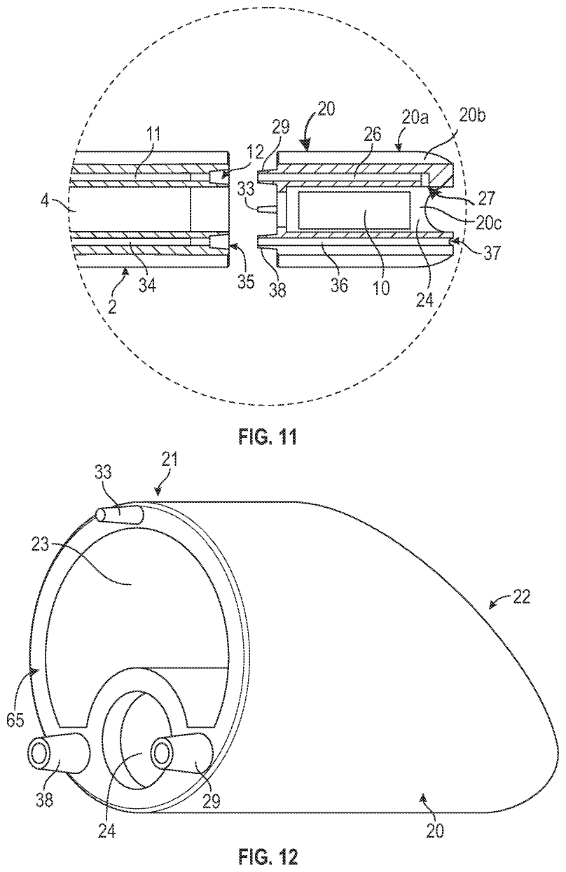

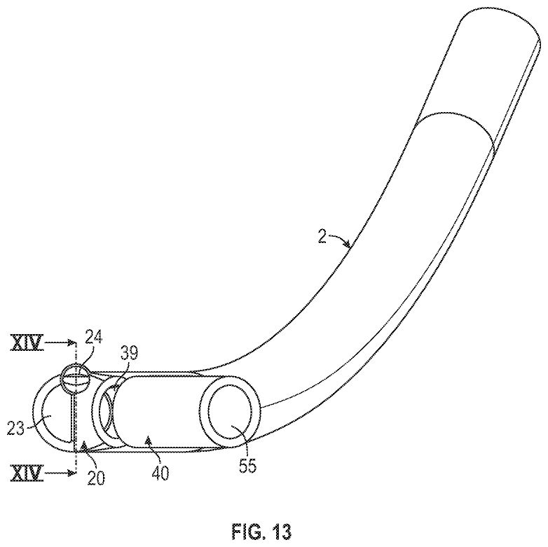

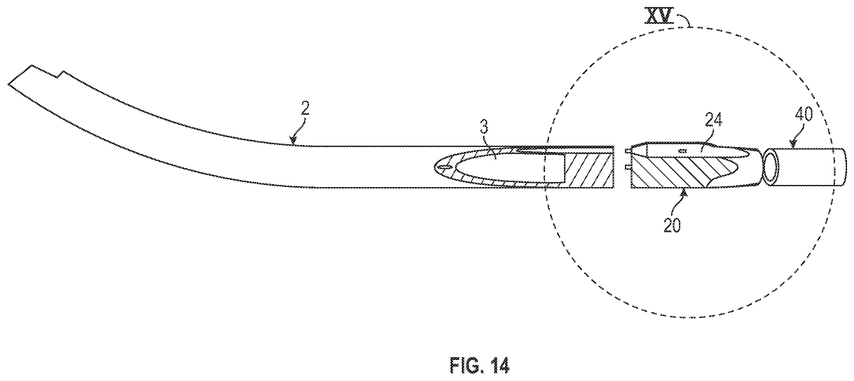

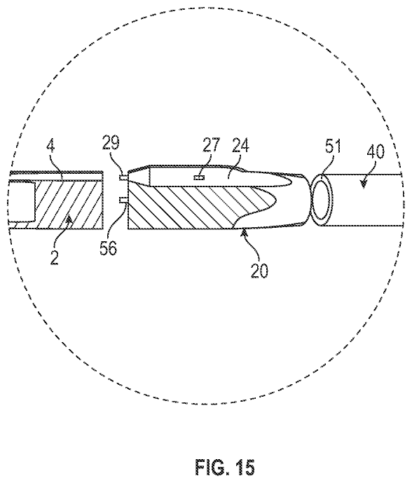

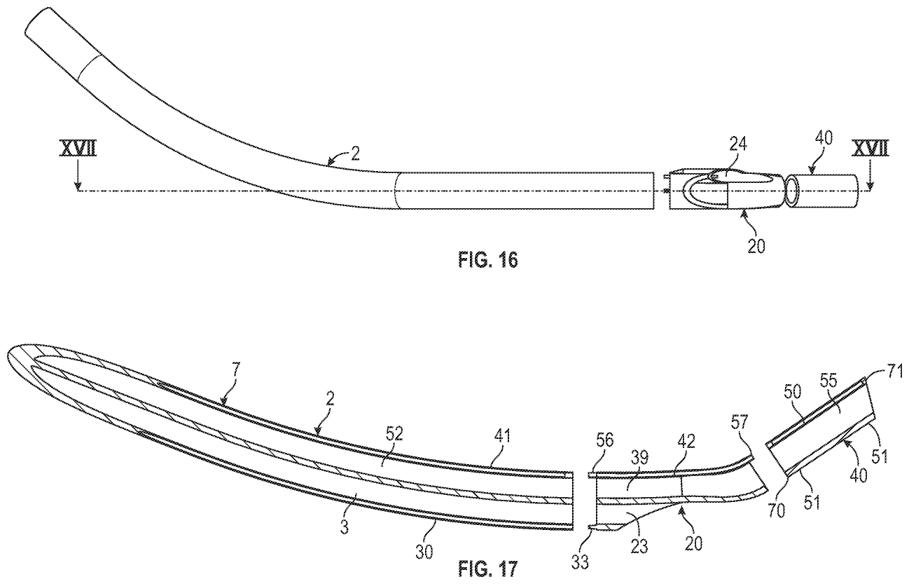

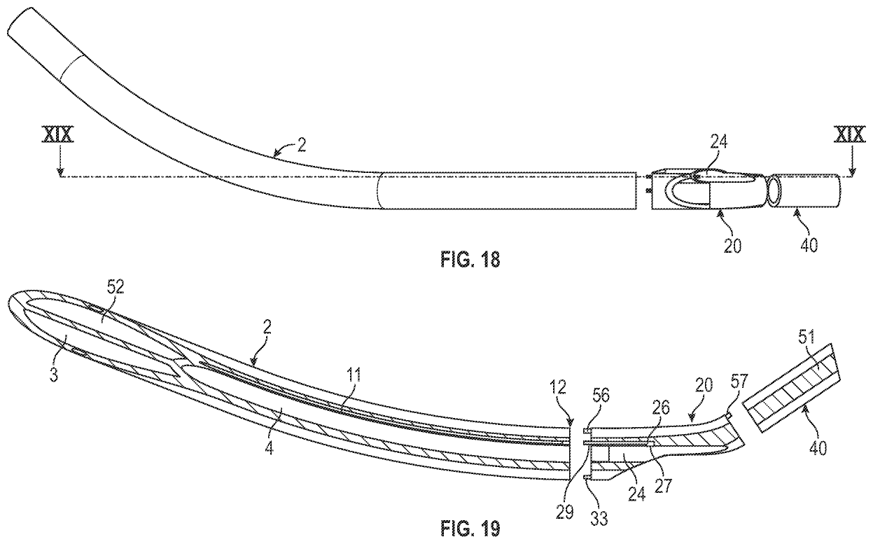

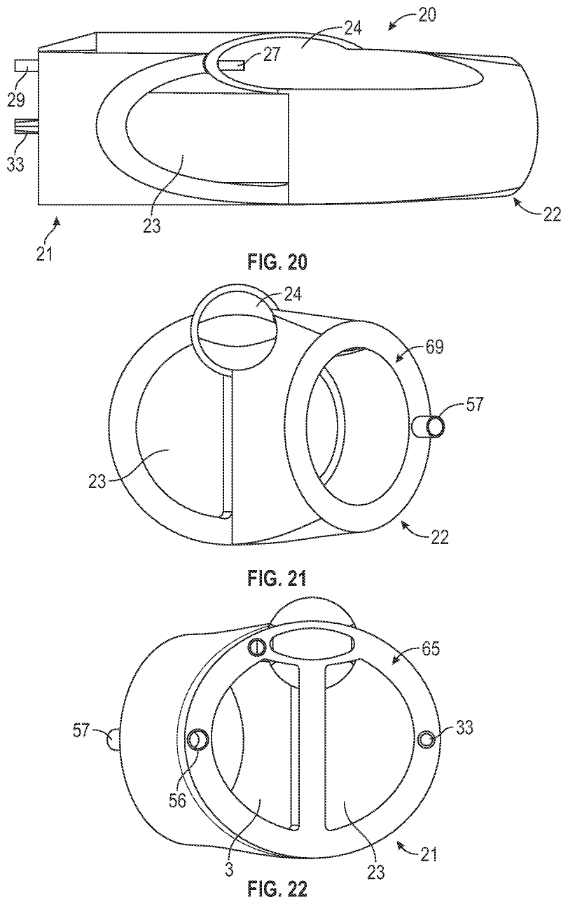

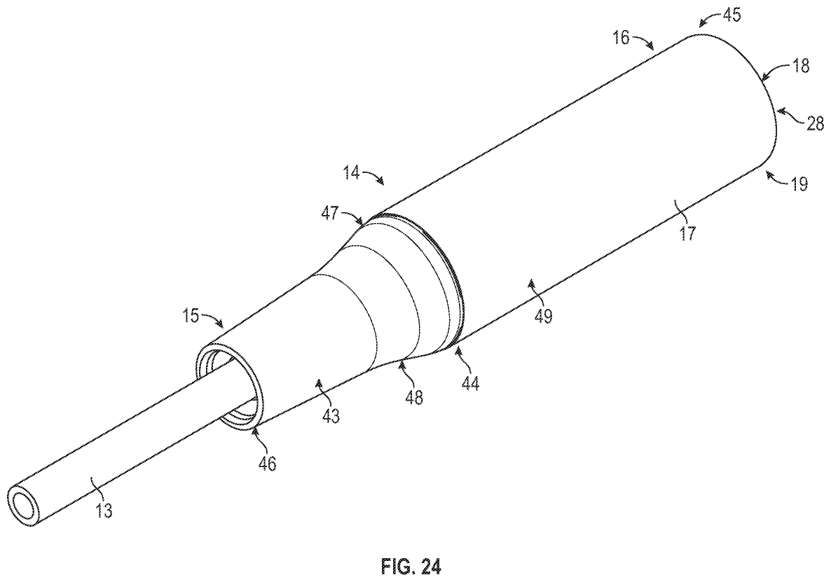

According to the present disclosure, the medical device 1 includes a tube 2 as illustrated in FIGS. 5 to 12. The tube has a first lumen 3 and a dedicated camera lumen 4. The first lumen 3 has a proximal end 5 and a distal end 6, the distal end 6 being adapted to be placed inside a body cavity during use of the medical device 1. The tube 2 has an outer wall 7 enclosing the first lumen 3 and the camera lumen 4, and the camera lumen 4 has a proximal end 8 and a distal end 9. A camera module 10 (shown in FIGS. 8 and 11) includes a not shown image sensor and one or more not shown light sources and is arranged in a camera lumen extension formed in a separately manufactured tip housing 20. The light sources may have the form of one or more LED's or incandescent bulbs. Furthermore, the light sources may be provided in the form of one or more fibre optic cables transmitting light from outside the camera module 10 to the camera module 10. An image transmission cable 13 (best seen in FIG. 24) is attached to the camera module 10 and extends through part of the camera lumen 4 formed in the tube 2 in order to, during use, connect to a monitor or image display device 68, directly or by means of a wireless connection. The image transmission cable 13 transmits the image data from the image sensor of the camera module 10, but furthermore, the image transmission cable 13 may transmit electric energy to an electric circuit of the camera module 10 and the light source of the camera module 10. Furthermore, the image transmission cable 13 may include one or more fibre optic cables transmitting light from outside the camera module 10 to the camera module 10.

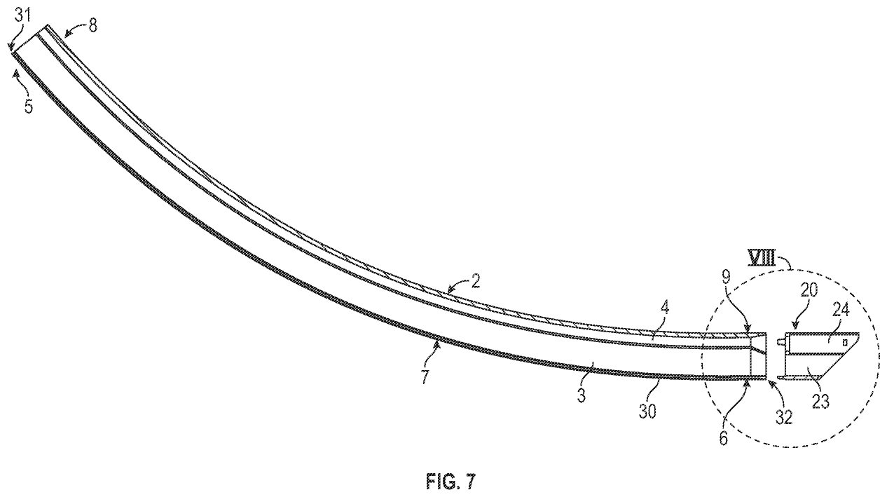

The medical device 1 according to the present disclosure furthermore includes a separately manufactured tip housing 20 (mentioned in the preceding paragraph) having a tubular housing part 20a extending between a proximal end 21 and a distal end 22, as seen in FIGS. 8-12. As illustrated in FIGS. 7 and 8, the proximal end 21 of the tip housing 20 is mounted on the main tube 2 at the distal end 6 of the first lumen 3. Thereby, the tip housing 20 forms a tip extension 23 of the first lumen 3 and a tip extension 24 of the camera lumen 4, which may be referred to, respectively, as the first lumen extension and the camera lumen extension. The tip extension 23 of the first lumen 3 has an open end 25, and the camera housing 14 (shown in FIG. 24) is arranged at least partly in the tip extension 24 of the camera lumen 4. However, in some embodiments, the camera housing 14 may extend somewhat into the camera lumen 4 of the main tube 2. Thereby, the distal end of the camera lumen 4 may be dilated to form a tapering end part 72.

By arranging the camera housing 14 in a tip housing 20 mounted as an extension on the main tube 2, the tip housing 20 may be manufactured separately by other manufacturing techniques than extrusion, thereby forming the tip extension 24 of the camera lumen 4 precisely for fast, easy and correct positioning of the camera module 10, and thereby forming other necessary details, such as flushing channels, without manual adaptation during assembly.

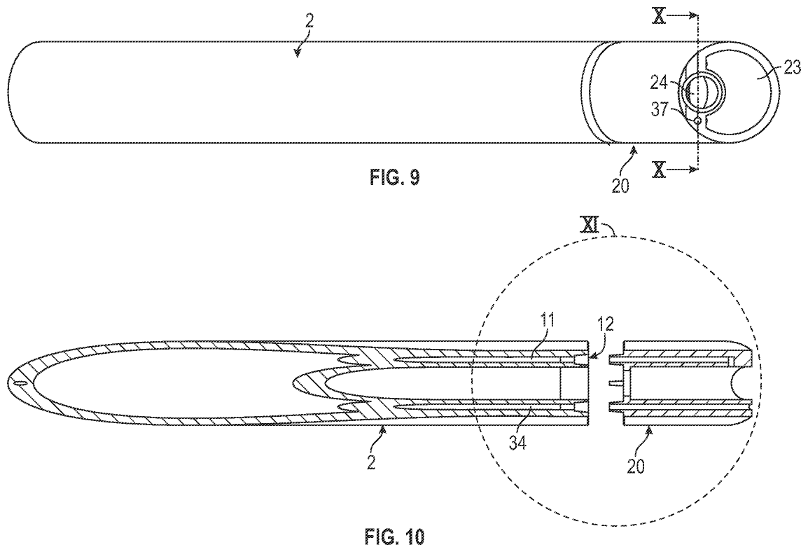

As illustrated in FIGS. 9 to 12, the main tube 2 has a flush lumen 11 enclosed by the outer wall 7, wherein the flush lumen 11 has a proximal end and a distal end 12. The tip housing 20 forms a tip extension 26 of the flush lumen 11 (also referred to as the flush lumen extension), and the tip extension 26 of the flush lumen 11 communicates with the flush outlet 27 directed at a lens end 28 of the camera housing 14. As shown in FIG. 11, the tubular housing part 20a comprises an outer surface 20b and an inner surface 20c. The camera module 10 may be fixed in the tip extension 24 of the camera lumen 4 by means of the camera housing 14, as illustrated in FIG. 24. The flush outlet 27 may form part of the camera housing 14 in that a channel in camera housing 14 may communicate with the tip extension 26 of the flush lumen 11 or may communicate directly with the flush lumen 11. Any suitable number of flush outlets 27 may be arranged and may be supplied with fluid through one common flush lumen 11 or more such flush lumens 11 arranged in the main tube 2. By arranging the flush outlet 27 or outlets as an integrated part of the camera housing 14, an even more efficient cleaning of the camera front may be obtained as a result of close and precise positioning of the flush outlet in relation to a front lens of the camera or a transparent wall behind which a camera lens stack is arranged.

As seen in FIG. 11, in the tip housing 20, the tip extension 26 of the flush lumen 11 is provided with a protruding pipe 29, also referred to as a flush pipe, engaging the flush lumen 11 of the main tube 2 in the mounted position of the tip housing 20 on the main tube 2. Thereby, a continuous lumen without blockage in the junction may be ensured. The protruding pipe 29 may be manufactured as an integrated part of the tip housing 2 or may be a separate pipe component inserted into the tip housing 20.

In an embodiment of the present disclosure, the main tube 2 of the medical device 1, as illustrated in FIG. 5, is provided with a first inflatable cuff 59 as illustrated in the prior art single lumen endotracheal tube 2 of FIG. 3. As seen in FIGS. 7 and 8, the main tube 2 has a first cuff lumen 30 being enclosed by the outer wall 7 and having a proximal end 31 and a distal end 32. The first cuff lumen 30 communicates with the first inflatable cuff 59 in a not shown manner known per se, i.e. through an opening through the outer wall 7. In the mounted position of the tip housing 20 on the main tube 2, the distal end 32 of the first cuff lumen 30 is closed by means of a sealing plug 33 arranged on the proximal end 21 of the tip housing 20. Thereby, the first inflatable cuff 59 may be arranged on the main tube 2 without having to separately close the distal end 32 of the first cuff lumen 30 during production. The sealing plug 33 may be a separate part arranged on the tip housing 20 or it may be an integral part of the tip housing 20.

Furthermore, in the embodiment illustrated in FIGS. 10 and 11, the main tube 2 has an anesthetic lumen 34 being enclosed by the outer wall 7 and having a proximal end and a distal end 35. The tip housing 20 forms a tip extension 36 of the anesthetic lumen 34 (also referred to as the anesthetic lumen extension), and the tip extension 36 of the anesthetic lumen 34 communicates with at least one anesthetic outlet port 37. The at least one anesthetic outlet port 37 may be precisely formed during manufacturing of the tip housing 20 without manual adaptation during production of the device. As seen in FIG. 11, the tip extension 36 of the anesthetic lumen 34 is provided with a proximally protruding pipe 38, also referred to as an anesthetic pipe, engaging the anesthetic lumen 34 of the main tube 2. The anesthetic outlet port 37 may allow administration of a local anesthetic to prevent pharyngeal (gag) reflex, which may be advantageous during awake intubation.

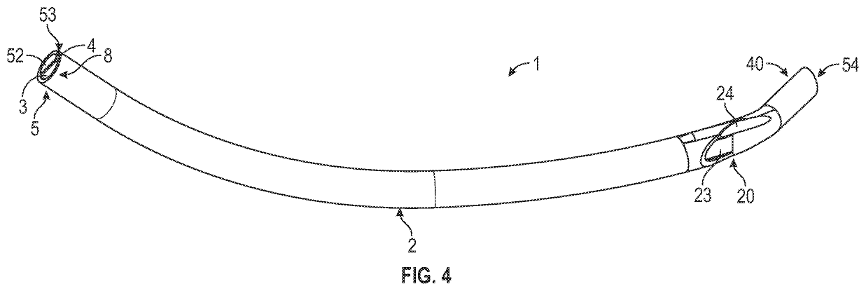

In the embodiment illustrated in FIG. 4, the main tube 2 has a second lumen 52 having a proximal end 53 and a distal end 54. The second lumen 52 is enclosed by the outer wall 7, and the tip housing 20 forms a tip extension 39 of the second lumen 52, as seen in FIG. 17. A distal tube 40 is mounted on the distal end 22 of the tip housing 20, thereby forming a further extension 55 of the second lumen 52. The distal tube 40 may have different properties than the main tube 2 whereby more design freedom may be achieved. The distal tube 40 may preferably be an extruded tube. For instance, the distal tube 40 may be more flexible than the main tube 2 and may at least be more flexible than the tip housing 20. However, alternatively, the distal tube 40 could be replaced by an integrated part of the tip housing 20. Such integrated part of the tip housing 20 and the tip housing itself could be produced together from two different materials, respectively, by a co-injection moulding technique, whereby the materials have different properties, such as different flexibilities.

In an embodiment of the present disclosure, the distal tube 40 of the medical device 1 as illustrated in FIG. 4 is provided with a second inflatable cuff 60 as illustrated in the prior art double lumen endotracheal tube 2 of FIGS. 1 and 2. As seen in FIG. 17, the main tube 2 has a second cuff lumen 41 enclosed by the outer wall 7. The tip housing 20 forms a tip extension 42 of the second cuff lumen 41, and the distal tube 40 forms a distal second cuff lumen 50 enclosed by an outer wall 51 of the distal tube 40. The distal second cuff lumen 50 of the distal tube 40 connects the tip extension 42 of the second cuff lumen 41 with the second inflatable cuff 60 in a not shown manner known per se, i.e. through an opening through the outer wall 51. An end opening 71 of the distal second cuff lumen 50 has been closed by means of a plug or the like.

As further seen in FIG. 17, the tip extension 42 of the second cuff lumen 41 is provided with a first protruding pipe 56 arranged at the proximal end 21 of the tip housing 20 and engaging the second cuff lumen 41 of the main tube 2. The tip extension 42 of the second cuff lumen 41 is also provided with a second protruding pipe 57 arranged at the distal end 22 of the tip housing 20 and engaging the distal second cuff lumen 50 of the distal tube 40. Thereby, a continuous lumen without blockage in the junctions may be ensured. The protruding pipes 56, 57 may be manufactured as integrated parts of the tip housing 20 or may be separate pipe components inserted into the tip housing.

According to the present disclosure, the camera module 10 may be fixed in the tip extension 24 of the camera lumen 4 by means of a camera housing 14 as illustrated in FIG. 24. The camera housing 14 extends in a longitudinal direction and has a proximal end 15 and a distal end 16. The camera housing 14 includes a tubular housing part 17 at least partly surrounding the camera module 10 and a distal end wall 18, and the tubular housing part 17 and the distal end wall 18 are integrally moulded and form one single housing element 19. The tubular housing part 17 fits tightly into the tip extension 24 of the camera lumen 4 of the tube 2.

Thereby, precise mounting of the camera module 10 in the tip extension 24 may be facilitated in that the tight fit between the tubular housing part 17 and the tip extension 24 may ensure precise positioning of the camera module 10 in the tip extension 24 without any adjustments being necessary. Although it may be preferred that the tubular housing part 17 has a circular cross-section as illustrated in the figures, any tubular form is possible. For instance, the cross-section of the tubular housing part 17 may have any suitable rounded form, such as oval or elliptic. The cross-section of the tubular housing part 17 may also have other forms, such as polygonal form, in particular a triangular, square, rectangular, pentagonal, hexagonal, heptagonal, or octagonal form or any suitable form.

Preferably, the tip housing 20 is made by moulding, such as injection moulding, or 3D printing. Thereby, it may be ensured that the tip extension 24 of the camera lumen 4 may be formed precisely for fast, easy and correct positioning of the camera module, and thereby also forming other necessary details, such as flushing channels, without manual adaptation during assembly.

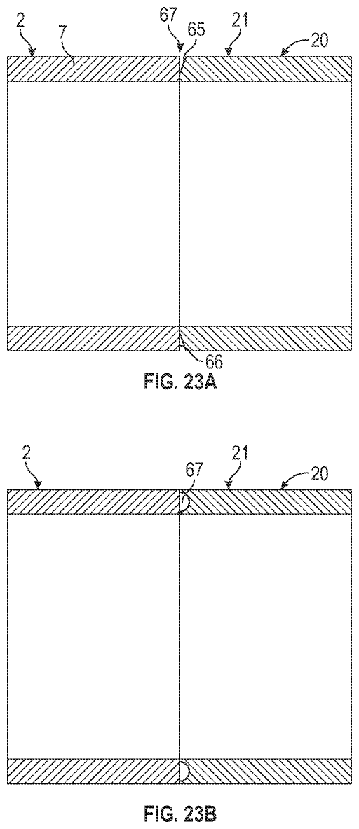

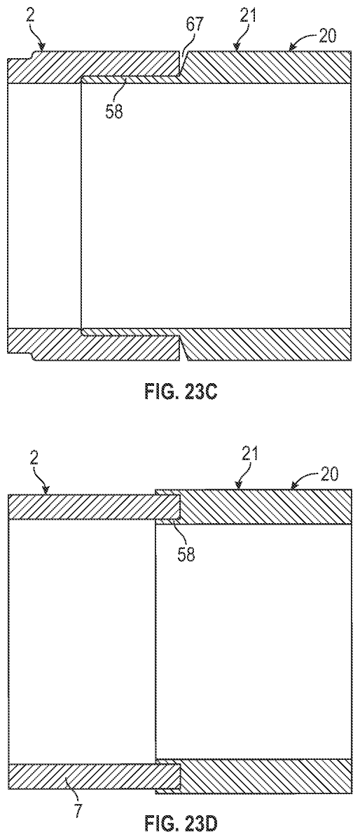

As illustrated in FIGS. 23A to 23D, the proximal end 21 of the tip housing 20 has a proximal junction interface 65 fitting an abutment surface 66 of the main tube 2. The abutment surface 66 includes at least an end face of the outer wall 7 of the main tube 2. In the embodiments illustrated in FIGS. 23A to 23C, the proximal junction interface 65 forms a groove 67 with said abutment surface 66 of the main tube 2. The groove 67 is adapted to take up glue or any applicable solvent in order to securely fuse together the housing 20 and the main tube 2. In the embodiment of FIG. 23C, the proximal end 21 of the tip housing 20 is provided with a protruding pipe 58 engaging the first lumen 3 and/or the second lumen 52, as well as possibly the camera lumen 4, of the main tube 2. In the embodiment of FIG. 23D, the proximal end 21 of the tip housing 20 is provided with a groove in which the outer wall 7 of the main tube 2 is inserted.

Comparing FIGS. 17 and 21, the distal end 22 of the tip housing 20 has a distal junction interface 69 fitting an abutment surface 70 on the proximal end of the distal tube 40. Said abutment surface 70 includes an end face of the outer wall 7 of the distal tube 40. In a similar way as illustrated in FIGS. 23A to 23D, the distal junction interface 69 may form a groove with said abutment surface 70 of the distal tube 40, wherein said groove may contain hardened glue. Furthermore in a similar way as illustrated in FIG. 23C, the tip extension 39 of the second lumen 52 may, at the distal end 22 of the tip housing 20, be provided with a protruding pipe engaging the further extension of the second lumen 52 formed in the distal tube 40.

According to the present disclosure, the medical device 1 is a catheter, preferably an airway device, and preferably an endotracheal tube. Furthermore according to the present disclosure, a system may include a medical device as described and including a monitor or image display device 68 connected to the image transmission cable 13 directly or via a wireless connection as seen in the prior art prior art double lumen endotracheal tube 2 illustrated in FIGS. 1 and 2.

The following items correspond to the original claims.
1. A medical device in the form of an airway device including a main tube having at least a first lumen and a dedicated camera lumen, the first lumen having a proximal end and a distal end, the distal end being adapted to be placed inside a body cavity during use of the medical device, the tube having an outer wall enclosing at least the first lumen and the camera lumen, the camera lumen having a proximal end and a distal end, a camera module including at least an image sensor and one or more light sources being arranged at the distal end of the first lumen, and an image transmission cable attached to the camera module extending through at least part of the camera lumen in order to connect to an image display device, wherein a separately manufactured tip housing has a proximal end and a distal end, wherein the proximal end of the tip housing is mounted on the main tube at the distal end of the first lumen, wherein the tip housing forms a tip extension of the first lumen and a tip extension of the camera lumen, wherein the tip extension of the first lumen has an open end, and wherein the camera module is arranged at least partly in the tip extension of the camera lumen.
2. A medical device according to item 1, wherein the main tube has a flush lumen enclosed by the outer wall, wherein the flush lumen has a proximal end and a distal end, wherein the tip housing forms a tip extension of the flush lumen, and wherein the tip extension of the flush lumen communicates with at least one flush outlet directed at a lens end of the camera module.
3. A medical device according to item 2, wherein the camera module is fixed in the tip extension of the camera lumen by means of a camera housing, and wherein the at least one flush outlet forms part of the camera housing.
4. A medical device according to item 2 or 3, wherein the tip extension of the flush lumen is provided with a proximally protruding pipe engaging the flush lumen of the main tube.
5. A medical device according to any one of the preceding items, wherein the main tube is provided with a first inflatable cuff, wherein the main tube has a first cuff lumen enclosed by the outer wall and communicating with the first inflatable cuff, wherein the first cuff lumen has a proximal end and a distal end, and wherein the distal end of the first cuff lumen is closed by means of a sealing plug arranged on the proximal end of the tip housing.
6. A medical device according to any one of the preceding items, wherein the main tube has an anesthetic lumen enclosed by the outer wall, wherein the anesthetic lumen has a proximal end and a distal end, wherein the tip housing forms a tip extension of the anesthetic lumen, and wherein the tip extension of the anesthetic lumen communicates with at least one anesthetic outlet port.
7. A medical device according to item 6, wherein the tip extension of the anesthetic lumen is provided with a proximally protruding pipe distally engaging the anesthetic lumen of the main tube.
8. A medical device according to any one of the preceding items, wherein the main tube has a second lumen having a proximal end and a distal end, wherein the second lumen is enclosed by the outer wall, wherein the tip housing forms a tip extension of the second lumen.
9. A medical device according to item 8, wherein a distal tube is mounted on the distal end of the tip housing, thereby forming a further extension of the second lumen.
10. A medical device according to item 9, wherein the distal tube is provided with a second inflatable cuff, wherein the main tube has a second cuff lumen enclosed by the outer wall, wherein the tip housing forms a tip extension of the second cuff lumen, wherein the distal tube forms a distal second cuff lumen enclosed by an outer wall of the distal tube, and wherein the distal second cuff lumen of the distal tube connects the tip extension of the second cuff lumen and the second inflatable cuff.
11. A medical device according to item 10, wherein the tip extension of the second cuff lumen is provided with a first protruding pipe arranged at the proximal end of the tip housing and engaging the second cuff lumen of the main tube, and wherein the tip extension of the second cuff lumen is provided with a second protruding pipe arranged at the distal end of the tip housing and engaging proximal end of the distal second cuff lumen of the distal tube.
12. A medical device according to any one of the preceding items, wherein the camera module is fixed in the tip extension of the camera lumen by means of a camera housing extending in a longitudinal direction and having a proximal end and a distal end, wherein the camera housing includes a tubular housing part at least partly surrounding the camera module and a distal end wall, wherein the tubular housing part and the distal end wall are integrally moulded and form one single housing element, and wherein the tubular housing part fits tightly into the tip extension of the camera lumen of the tube.
13. A medical device according to any one of the preceding items, wherein the tip housing is made by moulding, such as injection moulding, or 3D printing.
14. A medical device according to any one of the preceding items, wherein the proximal end of the tip housing has a proximal junction interface fitting an abutment surface of the main tube, wherein said abutment surface includes at least an end face of the outer wall of the main tube, wherein the proximal junction interface forms a groove with said abutment surface of the main tube, and wherein said groove contains hardened glue.
15. A medical device according to any one of the preceding items, wherein the tip extension of the at least first lumen, at the proximal end of the tip housing, is provided with a proximally protruding pipe engaging the distal end of the at least first lumen of the main tube.
16. A medical device according to any one of the items 9 to 11, wherein the distal end of the tip housing has a distal junction interface fitting an abutment surface of the distal tube, wherein said abutment surface includes at least an end face of the outer wall of the distal tube, wherein the distal junction interface forms a groove with said abutment surface of the distal tube, and wherein said groove contains hardened glue.
17. A medical device according to any one of the items 9 to 11 or 16, wherein the tip extension of the second lumen, at the distal end of the tip housing, is provided with a protruding pipe engaging the proximal end of the further extension of the second lumen formed in the distal tube.
18. A medical device according to any one of the preceding items, wherein the medical device is a catheter.
19. A medical device according to any one of the preceding items, wherein the camera module is fixed in the tip extension of the camera lumen.
20. A medical device according to any one of the preceding items, wherein the medical device is an endotracheal tube.
21. A system including a medical device according to any one of the preceding items, wherein the system includes a monitor or image display device connected to the image transmission cable directly or by means of a wireless connection.

### LIST OF REFERENCE NUMBERS

1 medical device
2 main tube
3 first lumen of tube
4 camera lumen
5 proximal end of first lumen
6 distal end of first lumen
7 outer wall of tube
8 proximal end of camera lumen
9 distal end of camera lumen
10 camera module
11 flush lumen of main tube
12 distal end of flush lumen of main tube
13 image transmission cable
14 camera housing
15 proximal end of camera housing
16 distal end of camera housing
17 tubular housing part of camera housing
18 distal end wall of camera housing
19 single housing element formed by tubular housing part and distal end wall
20 tip housing
21 proximal end of tip housing
22 distal end of tip housing
23 tip extension of first lumen
24 tip extension of camera lumen
25 open end of tip extension of first lumen
26 tip extension of flush lumen
27 flush outlet
28 lens end of camera housing
29 protruding pipe of tip extension of flush lumen
first cuff lumen of main tube
31 proximal end of first cuff lumen
32 distal end of first cuff lumen
33 sealing plug of tip housing
34 anesthetic lumen of main tube
distal end of anesthetic lumen of main tube
36 tip extension of anesthetic lumen
37 anesthetic outlet port
38 protruding pipe of tip extension of anesthetic lumen
39 tip extension of second lumen of main tube
distal tube
41 second cuff lumen of main tube
42 tip extension of second cuff lumen
43 proximal portion of camera housing
44 proximal end of tubular housing part
distal end of tubular housing part
46 proximal end of proximal portion
47 distal end of proximal portion
48 tapering part of proximal portion
49 outer cylindrical surface of tubular housing part
distal second cuff lumen of distal tube
51 outer wall of distal tube
52 second lumen of main tube
53 proximal end of second lumen
54 distal end of second lumen
further extension of second lumen
56 first protruding pipe of tip extension of second cuff lumen
57 second protruding pipe of tip extension of second cuff lumen
58 protruding pipe of tip extension of first lumen
59 first inflatable cuff
60 second inflatable cuff
61a, 61b pilot balloon with one-way valve
62 flush connection
63 flush opening
64 flush channel
65 proximal junction interface of tip housing
66 abutment surface of main tube
67 groove
68 monitor or image display device
69 distal junction interface of distal end of tip housing
70 abutment surface of distal tube
71 end opening of distal second cuff lumen
72 tapering end part of camera lumen

1-21. (canceled)

22. A medical device including:
a main tube having an outer wall, a distal end, a first lumen, a flush lumen and a camera lumen, the first lumen having a proximal end and a distal end, the camera lumen having a proximal end and a distal end, the outer wall enclosing the first lumen, the camera lumen extending within the outer wall, and the flush lumen extending within the outer wall;
a tip housing separately manufactured from the main tube, the tip housing comprising a proximal end, a distal end, a tubular housing part extending from the proximal end to the distal end, a first lumen extension having an open end, a camera lumen extension, a flush lumen extension and a flush outlet configured to discharge fluid received from the flush lumen through the flush lumen extension, the first lumen extension extending distally from the first lumen, the camera lumen extension extending distally from the camera lumen, the flush lumen extension axially aligned with and extending distally from the flush lumen, the tubular housing part enclosing the first lumen extension and the camera lumen extension, the flush lumen extension extending between an outer surface and an inner surface of the tubular housing part with the flush outlet extending between the flush lumen extension and the inner surface, and the proximal end of the tip housing mounted at the distal end of the main tube;
a camera module arranged at least partly in the camera lumen extension of the tip housing, the flush outlet located distally of the camera module; and
a camera housing affixing the camera module in the camera lumen extension.

23. The medical device of claim 22, wherein the tubular housing part comprises a proximal end and a distal end, wherein the tip housing comprises a flush pipe aligned with the flush lumen extension and extending proximally of the proximal end of the tubular housing part, the flush pipe configured to engage the flush lumen of the main tube.

24. The medical device of claim 22, wherein the main tube has a second lumen having a proximal end and a distal end, wherein the second lumen is enclosed by the outer wall, and wherein the tip housing comprises a second lumen extension axially aligned and extending distally from the second lumen.

25. The medical device of claim 24, further comprising a distal tube mounted onto the distal end of the tip housing, wherein the distal tube and the tip housing comprise different materials.

26. A medical device including:
a main tube having an outer wall, a distal end, a first lumen and a camera lumen, the first lumen having a proximal end and a distal end, the camera lumen having a proximal end and a distal end, the outer wall enclosing the first lumen, and the camera lumen extending within the outer wall;
a tip housing separately manufactured from the main tube, the tip housing comprising a proximal end, a distal end, a tubular housing part extending from the proximal end to the distal end, a first lumen extension having an open end, and a camera lumen extension, the first lumen extension extending distally from the first lumen, the camera lumen extension extending distally from the camera lumen, the tubular housing part enclosing the first lumen extension and the camera lumen extension, and the proximal end of the tip housing mounted at the distal end of the main tube; and
a camera module arranged at least partly in the camera lumen extension of the tip housing.

27. The medical device of claim 26, further comprising a camera housing affixing the camera module in the camera lumen extension.

28. The medical device of claim 27, wherein the camera housing extends in a longitudinal direction and comprises a proximal end and a distal end, wherein the camera housing includes a tubular housing part at least partly surrounding the camera module and a distal end wall, wherein the tubular housing part and the distal end wall are integrally moulded and form one single housing element, and wherein the tubular housing part fits tightly into the camera lumen extension.

29. The medical device of claim 26, wherein the main tube comprises a flush lumen enclosed by the outer wall, wherein the tip housing comprises a flush lumen extension aligned with and extending distally from the flush lumen, the tubular housing part enclosing the flush lumen extension and comprising a flush outlet configured to discharge fluid received from the flush lumen through the flush lumen extension.

30. The medical device of claim 29, wherein the flush lumen is intermediate the proximal end and the distal end of the tip housing and configured to discharge flushing fluid distally of a distal end of the camera housing.

31. The medical device of claim 29, wherein the tubular housing part comprises a proximal end and a distal end, wherein the tip housing comprises a flush pipe aligned with the flush lumen extension and extending proximally of the proximal end of the tubular housing part, the flush pipe configured to engage the flush lumen of the main tube.

32. The medical device of claim 26, wherein the main tube comprises an anesthetic lumen enclosed by the outer wall, wherein the tip housing comprises an anesthetic lumen extension aligned with and extending distally from the anesthetic lumen, wherein the tip housing comprises an anesthetic outlet port, and wherein the anesthetic lumen extension is in fluid communication with the anesthetic outlet port to discharge an anesthetic received from the anesthetic lumen through the anesthetic lumen extension.

33. The medical device of claim 32, wherein the tip housing comprises an anesthetic pipe aligned with the anesthetic lumen extension and extending proximally of the proximal end of the tubular housing part, the anesthetic pipe configured to engage the anesthetic lumen of the main tube.

34. The medical device of claim 26, wherein the main tube comprises a second lumen having a proximal end and a distal end, wherein the second lumen is enclosed by the outer wall, and wherein the tip housing comprises a second lumen extension axially aligned and extending distally from the second lumen.

35. The medical device of claim 34, further comprising a distal tube mounted onto the distal end of the tip housing.

36. The medical device of claim 35, wherein the distal tube is provided with a second inflatable cuff, wherein the main tube has a second cuff lumen enclosed by the outer wall, wherein the tip housing forms a tip extension of the second cuff lumen, wherein the distal tube forms a distal second cuff lumen enclosed by an outer wall of the distal tube, and wherein the distal second cuff lumen of the distal tube connects the tip extension of the second cuff lumen and the second inflatable cuff.

37. The medical device of claim 36, wherein the tip extension of the second cuff lumen is provided with a first protruding pipe arranged at the proximal end of the tip housing and engaging the second cuff lumen of the main tube, and wherein the tip extension of the second cuff lumen is provided with a second protruding pipe arranged at the distal end of the tip housing and engaging proximal end of the distal second cuff lumen of the distal tube.

38. The medical device of claim 35, wherein the distal end of the tip housing has a distal junction interface fitting an abutment surface of the distal tube, wherein said abutment surface includes at least an end face of the outer wall of the distal tube, wherein the distal junction interface forms a groove with said abutment surface of the distal tube, and wherein said groove contains hardened glue.

39. The medical device of claim 36, wherein the second lumen extension, at the distal end of the tip housing, is provided with a protruding pipe engaging the proximal end of the further extension of the second lumen formed in the distal tube.

40. The medical device of claim 26, wherein the proximal end of the tip housing has a proximal junction interface fitting an abutment surface of the main tube, wherein said abutment surface includes at least an end face of the outer wall of the main tube, wherein the proximal junction interface forms a groove with said abutment surface of the main tube, and wherein said groove contains hardened glue.

41. The medical device of claim 26, wherein the first lumen extension, at the proximal end of the tip housing, is provided with a proximally protruding pipe engaging the distal end of the first lumen.

42. A system including a medical device according to claim 26 and a monitor or image display device communicatively connectable to the medical device.
